(19) United States
(12) Patent Application Publication
(10) Pub. No.: US 2020/0297440 A1
(43) Pub. Date: Sep. 24, 2020

(54) INTERACTIVE ANATOMICAL POSITIONER AND A ROBOTIC SYSTEM THEREWITH

(71) Applicant: THINK SURGICAL, INC., Fremont, CA (US)

(72) Inventor: Micah Forstein, Fremont, CA (US)

(73) Assignee: THINK SURGICAL, INC., Fremont, CA (US)

(21) Appl. No.: 16/649,218

(22) PCT Filed: Sep. 20, 2018
(86) PCT No.: PCT/US2018/051990

§ 371 Date: Mar. 20, 2020

Related U.S. Application Data
(60) Provisional application No. 62/561,061, filed on Sep. 20, 2017.

(51) Int. Cl. A61B 34/32; A61G 13/00; A61B 34/20; A61G 13/12; A61B 34/10

(52) U.S. Cl. CPC A61B 34/32; A61G 13/0063; A61B 34/20; A61G 13/1285; A61B 34/10; A61B 2034/105; A61B 2034/2068; A61B 2034/2055; A61B 2034/2051; A61B 2034/104; A61G 13/125

(57) ABSTRACT

A system and method are provided for improving the positioning of a target anatomy relative to a robot to perform a medical procedure. The system and method is especially advantageous for complex procedures that require a large operational workspace and/or several manipulator orientation changes to execute a surgical plan in its entirety such as total knee arthroplasty (TKA), as well as any of a wide variety of other surgical procedures, orthopedic or otherwise, including hip arthroplasty, ligament reconstruction, and shoulder arthroplasty. Dynamic and controlled repositioning of the target anatomy promotes robotic surgical system access in a way that any static positioning of the target anatomy cannot thereby speeding a surgical process and extending the spatial range of operation of the robot and tools carried thereon.

### RELATED APPLICATIONS

This application claims priority benefit of U.S. Provisional Application Ser. No. 62/561,064 filed 20 Sep. 2017; the contents of which are hereby incorporated by reference.

### FIELD OF THE INVENTION

The present invention generally relates to the field of robotic and computer assisted surgery; and in particular, to a new and useful process and system for dynamically positioning a bone relative to a surgical robot based on workspace and task requirements.

### BACKGROUND OF THE INVENTION

Robotic surgical procedures have become a preferred medical technique for many complex surgeries that require a high level of precision. Computer assisted surgical devices such as robots are gaining popularity as a tool to pre-operatively plan and precisely execute the plan to ensure an accurate final position and orientation of a prosthetic within a patient's bone that can improve long term clinical outcomes and increase the survival rate of a prosthesis compared to a manually performed surgery.

Common surgical procedures performed with robotic assistance are total and partial joint replacements. For example, total knee arthroplasty (TKA) is a surgical procedure in which the articulating surfaces of the knee joint are replaced with prosthetic components, or implants. TKA requires the removal of worn or damaged bone/cartilage on the distal femur and proximal tibia. The removed bone is then replaced with synthetic implants, typically formed of metal or plastic, to create new joint surfaces.

The position and orientation (POSE) of the removed bone, referred to as bone cuts or resected bone, predominantly determines the final placement of the implants within the joint. Generally, in TKA, a surgeon plans and creates the bone cuts so the final placement of the implants restores the mechanical axis or kinematics of the patient's leg while preserving the balance of the surrounding knee ligaments. Creating the bone cuts to correctly align the femoral knee implant is especially difficult because the femur typically requires at least five planar bone cuts. Any malalignment in any one of these planar cuts or orientations may have drastic consequences on the final result of the procedure and the wear pattern of the implant, resulting in reduced functionality and decreased implant longevity.

To ensure correct resectioning of the planar cuts during a TKA procedure, a robotic surgical system, such as the TSolution One™ Surgical System (THINK Surgical, Fremont, Calif.) is used to precisely mill the planar cuts according to a pre-operative plan. In order to accurately execute the surgical plan on the bones of the joint with a robotic system, the correct positioning of the robot with respect to the target anatomy is critical. Currently, a moveable base of the robotic system is manually maneuvered next to the target anatomy and fixed into a position using a braking mechanism on the moveable base. With reference to FIG. 1, a knee positioning apparatus 100 as described in U.S. Pat. No. 7,380,299 to DeMayo, manually secures the distal end of the patient's leg relative to the moveable base. The knee positioning apparatus 100 allows a user to manually position and secure the anatomy relative to the surgical system. The position and orientation (POSE) of the bone is then registered to the surgical plan and surgical system using a tracked device by a tracking system (e.g., an optical tracking system, a mechanical tracking system) and registration techniques known in the art. After the respective coordinates are known, the robotic system determines if an end-effector tool of the robot can perform a surgical task (i.e., execute a surgical plan) on the anatomy. All of the points or boundaries defined relative to the anatomy in the task or surgical plan should be within the workspace of the robot and hence reachable by the robot, also referred to herein as task requirements. If the robotic system determines the end-effector tool is unable to perform the surgical task within the workspace, the base or bone may need to be repositioned and the bone re-registered. There is also the possibility that when the position of the base with respect to the anatomy changes, this causes the designated points or boundaries to also change within the workspace of the robot. Some of these points or boundaries may be pushed out of the workspace and become unreachable. All of these problems can greatly increase the time needed to perform the operation, especially if the bone needs to be fixed to the robotic system (e.g., see robotic-bone fixation as described in U.S. Pat. No. 5,086,401).

In addition, there are other parameters of the robotic system that should be addressed prior to positioning the base and bone relative to one another. This may include how a manipulator arm of the robot articulates to perform the task (e.g., robot orientations that avoid singularities, robot orientations that reduce vibrations, robot orientations that improve milling speed), also referred to herein as manipulator requirements. There may be preferences by a surgeon or a surgical team to have particular access points or corridors to the operating site that the robotic system may otherwise interfere, also referred to herein as user preferences. If an optical tracking system is present, the base should be positioned to maintain the line-of-sight between any tracking markers and the tracking system throughout the entire procedure. By simply guessing a position for the anatomy next to the surgical system, these parameters will not be optimal.

Thus, there exists a need for a system and method to optimally position or reposition a patient's target anatomy relative to a robotic system according to task requirements, manipulator requirements, or user preferences to execute a surgical plan.

### SUMMARY OF THE INVENTION

A robotic surgical system is provided for performing a surgical procedure on a target anatomy of a subject. The robotic surgical system includes a computing system configured to generate surgical plan data, a surgical robot having a movable base connected to a manipulator arm and adapted to hold an operating tool, where the surgical robot receives positional information associated with the surgical plan data from the computing system; and an active anatomical positioning device having an anatomical attachment region adapted to couple to the subject and in communication with a controller. The anatomical positioning device is adapted to automatically position the target anatomy relative to the surgical robot in response to instructions from the computing system.

A method is provided for positioning a target anatomy of a subject in an optimal position relative to the robotic surgical device described above. The method includes: determining a surgical plan based on surgical plan data, the surgical plan having a task to be executed on the target anatomy; coupling the subject proximal to the target anatomy to said anatomical attachment region; evaluating an initial position of the target anatomy relative to said robotic surgical system; determining the optimal position for the target anatomy based on the initial position and the task; and positioning the target anatomy in the optimal position by adjusting a position of the boot with the active anatomical positioning device relative to the initial position to the optimal position prior to executing the task.

### BRIEF DESCRIPTION OF THE DRAWINGS

Examples illustrative of embodiments are described below with reference to figures attached hereto. In the figures, identical structures, elements or parts that appear in more than one figure are generally labeled with a same numeral in all the figures in which they appear. Dimensions of components and features shown in the figures are generally chosen for convenience and clarity of presentation and are not necessarily shown to scale.

The subject matter that is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 depicts a prior art example of a knee positioning apparatus;

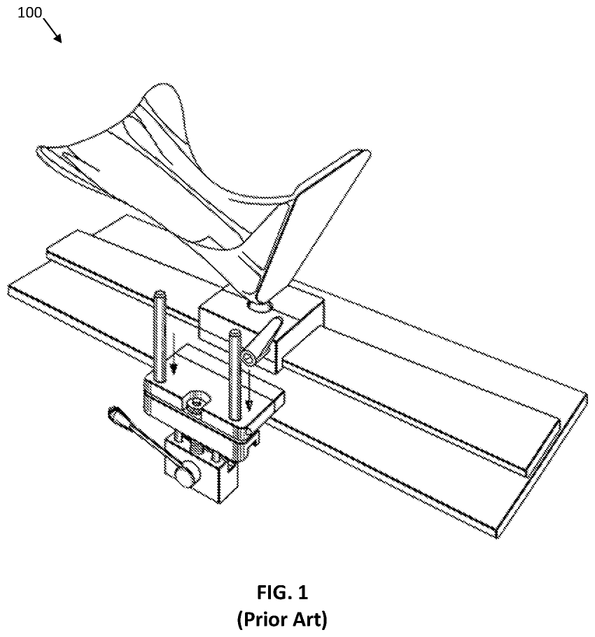

FIG. 2 is a flowchart depicting a method for automatically positioning a leg to the optimum position during a surgical procedure in accordance with embodiments of the invention;

FIG. 3 is a flowchart depicting a method for automatically positioning a leg to the optimum position during a surgical procedure in accordance with embodiments of the invention;

FIGS. 4A-4B illustratively depict a knee positioning apparatus with mounting elements in accordance with embodiments of the invention;

FIG. 5 illustrates a subject's leg positioned and secured in a knee positioning apparatus with a securement having a fixed fiducial array attached in accordance with embodiments of the invention;

FIG. 6 illustrates a subject's leg positioned and secured in a knee positioning apparatus with a securement in accordance with embodiments of the invention; and

FIG. 7 illustrates a subject's leg positioned and secured in a knee positioning apparatus wile attached to a surgical system in accordance with embodiments of the invention.

### DESCRIPTION OF THE INVENTION

The present invention has utility as a system and method for improving the positioning of a target anatomy relative to a robot to perform a medical procedure. The system and method is especially advantageous for complex procedures that require a large operational workspace and/or several manipulator orientation changes to execute a surgical plan in its entirety such as total knee arthroplasty (TKA). It should be appreciated that while the systems and methods described herein teach the automatic positioning of a bone to an optimal position in knee arthroplasty, any of a wide variety of other surgical procedures, orthopedic or otherwise, may likewise be used according to the teaching of this invention (e.g., hip arthroplasty, ligament reconstruction, shoulder arthroplasty). Dynamic and controlled repositioning of the target anatomy promotes robotic surgical system access in a way that any static positioning of the target anatomy cannot thereby speeding a surgical process and extending the spatial range of operation of the robot and tools carried thereon.

The present invention will now be described with reference to the following embodiments. As is apparent by these descriptions, this invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment may be deleted from that embodiment. In addition, numerous variations and additions to the embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations and variations thereof.

It is to be understood that in instances where a range of values are provided that the range is intended to encompass not only the end point values of the range but also intermediate values of the range as explicitly being included within the range and varying by the last significant figure of the range. By way of example, a recited range of from 1 to 4 is intended to include 1-2, 1-3, 2-4, 3-4, and 1-4.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference in their entirety.

### Definitions

Unless indicated otherwise, explicitly or by context, the following terms are used herein as set forth below.

As used in the description of the invention and the appended claims, the singular forms “a,” “an” and “the” are intended to include the plural forms as well, unless the context clearly indicates otherwise.

Also, as used herein, “and/or” refers to and encompasses any and all possible combinations of one or more of the associated listed items, as well as the lack of combinations when interpreted in the alternative (“or”).

As used herein, the term “digitizer” refers to a measuring device capable of measuring physical coordinates in three-dimensional space. For example, the ‘digitizer’ may be: a mechanical digitizer having passive links and joints, such as the high-resolution electro-mechanical sensor arm described in U.S. Pat. No. 6,033,415; a non-mechanically tracked digitizer probe (e.g., optically tracked, electromagnetically tracked, acoustically tracked, and equivalents thereof) as described for example in U.S. Pat. No. 7,043,961; or an end-effector of a robotic device.

As used herein, the term “digitizing” refers to the collecting, measuring, and/or recording of physical points in space with a digitizer.

As used herein, the term “pre-operative bone data” refers to bone data used to pre-operatively plan a procedure before making modifications to the actual bone. The pre-operative bone data may include one or more of the following: a patient's actual exposed bone prior to modification; an image data set of a bone; a virtual generic bone model; a physical bone model; a virtual patient-specific bone model; or a set of data collected directly on a bone intra-operatively commonly used with imageless computer-assist devices.

As used herein, the term “registration” refers to the determination of the POSE and/or coordinate transformation between two or more objects or coordinate systems such as a computer-assist device, a bone, pre-operative bone data, surgical planning data (i.e., an implant model, cut-file, virtual boundaries, virtual planes, or other cutting parameters or tasks associated with or defined relative to the pre-operative bone data), and any external landmarks (e.g., a fiducial marker array) associated with the bone, if such landmarks exist. Methods of registration are well known in the art and illustratively include the methods described in U.S. Pat. Nos. 6,033,415, 8,010,177, and 8,287,522.

As used herein, the term “optimal position” refers to a position of a target anatomy relative to a robot that satisfies at least one of task requirements, manipulator requirements, or user preferences to execute a surgical plan.

While the present invention is illustrated visually hereafter with respect to a femur as an example of the target anatomy for which the present invention is applied, it is appreciated that the present invention is equally applicable to other bones of a human, non-human primate, or other mammals.

Embodiments of the present invention describe a method and system for automatically and interactively positioning a target anatomy to an optimal position intra-operatively relative to a surgical device. Once an optimal position is determined, as further described below, information, recommendations, manual instructions, or automatic instructions may be provided to a user or anatomical positioning device to adjust the placement of the knee into that optimal position. In specific inventive embodiments, an active anatomical positioning device is provided to continuously or intermittently position the patient, automatically and in real-time, towards the optimal position relative to the robot to satisfy at least one of task requirements, manipulator requirements, or user preferences to throughout the execution of a surgical plan. As used herein, real-time denotes updating at between 1 and 5000 times per second, and in some embodiments, every 500 milliseconds (ms), every 100 ms, or every millisecond. It is appreciated that the rate of positional updating can be dynamic with more frequent updating occurring, for example, immediately prior to, or during task execution.

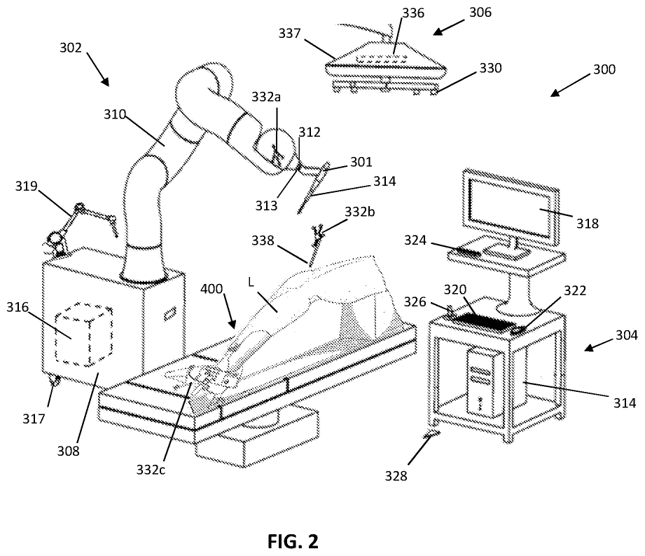

The robotic surgical systems described herein are generally capable of executing a surgical plan on a bone. Examples of such robotic surgical systems that may be augmented with an anatomical positioning device described herein include the TSolution One Surgical System (THINK Surgical, Inc., Fremont, Calif.), the RIO Robotic Arm Interactive Orthopedic System (Stryker-Mako, Ft. Lauderdale, Fla.) as described in U.S. Pat. No. 8,010,180; the ROSA Surgical System (Zimmer-Biomet, Warsaw, Ind.) as described in U.S. Pat. No. 9,237,861; as well as other autonomous or haptic serial-chain manipulators, parallel manipulators, or master-slave robotic systems. With reference to FIG. 2, a particular embodiment of a robotic surgical system 300 having an active anatomical positioning device 400 is shown. The surgical system 300 generally includes a surgical robot 302, a computing system 304, an active anatomical positioning device 400, and may include at least one of a mechanical digitizer arm 319 or a non-mechanical tracking system 306.

The surgical robot 302 may include a movable base 308, a manipulator arm 310 connected to the base 308, an end-effector flange 312 located at a distal end of the manipulator arm 310, and an end-effector assembly 301 for holding and/or operating a tool 314 removably attached to the flange 312 by way of an end-effector mount 313. The base 308 may include an actuator to adjust the height of the robotic arm 310. The base may further include a set of wheels 317 to maneuver the base 308, which may be fixed into position using a braking mechanism such as a hydraulic brake. The manipulator arm 310 includes various joints and links to manipulate the tool 314 in various degrees of freedom. The joints are illustratively prismatic, revolute, spherical, or a combination thereof. If a mechanical digitizer 319 or optical tracking system 306 is not present, the tool 314 may be a probe to act as a digitizer.

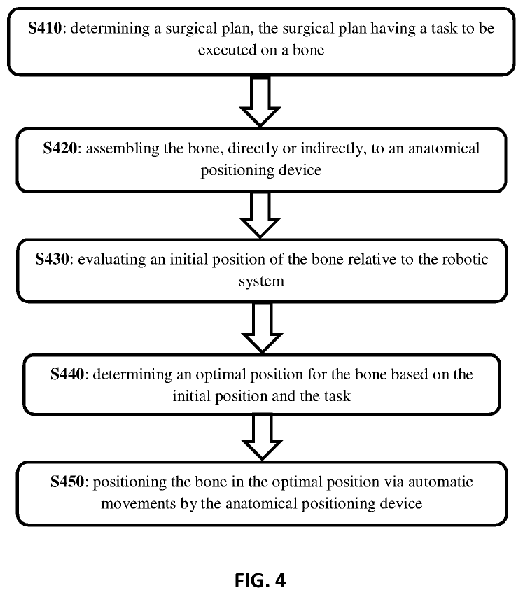

The computing system 304 generally includes a planning computer 314; a device computer 316; a positioning device computer/controller 416 (as shown in FIG. 4); a tracking computer 336 if a tracking system 306 is present; and peripheral devices. The planning computer 314, device computer 316, positioning device computer 416, and tracking computer 336, may be separate entities, single units, or combinations thereof depending on the surgical system. The peripheral devices allow a user to interface with the surgical system components and may include: one or more user-interfaces, illustratively including a display or monitor 318 having a graphical user interface; and user-input mechanisms, such as a keyboard 320, mouse 322, pendent 324, joystick 326, foot pedal 328, or the monitor 318 in some inventive embodiments has touchscreen capabilities.

The planning computer 314 contains hardware (e.g., processors, controllers, and/or memory), software, data, and utilities that are in some inventive embodiments dedicated to the planning of a surgical procedure, either pre-operatively or intra-operatively. This may include reading medical imaging data, segmenting imaging data, constructing three-dimensional (3D) virtual models, storing computer-aided design (CAD) files, providing various functions or widgets to aid a user in planning the surgical procedure, and generating surgical plan data. The final surgical plan includes operational task data for modifying a volume of tissue that is defined relative to the anatomy, such as a set of points in a cut-file to autonomously modify the volume of bone, a set of virtual boundaries defined to haptically constrain a tool within the defined boundaries to modify the bone, a set of planes or drill holes to drill pins in the bone, or a graphically navigated set of instructions for modifying the tissue. The data generated from the planning computer 314 may be transferred to the device computer 316 and/or tracking computer 336 through a wired or wireless connection in the operating room (OR); or transferred via a non-transient data storage medium (e.g., a compact disc (CD), a portable universal serial bus (USB) drive) if the planning computer 314 is located outside the OR.

The device computer 316 in some inventive embodiments is housed in the moveable base 308 and contains hardware, software, data, and utilities that are preferably dedicated to the operation of the surgical device 302. This may include surgical device control, robotic manipulator control, the processing of kinematic and inverse kinematic data, the execution of registration algorithms, the execution of global and local workspace algorithms, the execution of calibration routines, the execution of surgical plan data, coordinate transformation processing, providing workflow instructions to a user, and utilizing position and orientation (POSE) data from the tracking system 306.

The positioning device computer/controller 416 may include hardware, software, data, and utilities for controlling the anatomical positioning device 400. This may include the processing of forward and inverse kinematic data of the positioning device, receiving and processing tracking data and/or registration data, as well as having communication capabilities with at least one of the device computer 316, planning computer 314, or tracking computer 336. It is further contemplated that the positioning device 400 may be directly controlled via commands from the device computer 316 or tracking computer 336, without the need for a positioning device computer 416.

The optional tracking system 306 of the surgical system 300 may include two or more optical receivers 330 to detect the position of fiducial markers (e.g., retroreflective spheres, active light emitting diodes (LEDs)) uniquely arranged on rigid bodies. The fiducial markers arranged on a rigid body are collectively referred to as a fiducial marker array 332, where each fiducial marker array 332 has a unique arrangement of fiducial markers, or a unique transmitting wavelength/frequency if the markers are active LEDs to distinguish one marker array from another. An example of an optical tracking system is described in U.S. Pat. No. 6,061,644. The tracking system 306 may be built into a surgical light 337, located on a boom, a stand, or built into the walls or ceilings of the OR. The tracking system computer 336 may include tracking hardware, software, data, and utilities to determine the POSE of objects (e.g., bones B, surgical device 302) in a local or global coordinate frame. The POSE of the objects is collectively referred to herein as POSE data, where this POSE data may be communicated to the device computer 316 through a wired or wireless connection. Alternatively, the device computer 316 may determine the POSE data using the position of the fiducial markers detected from the optical receivers 330 directly.

The POSE data is determined using the position data detected from the optical receivers 330 and operations/processes such as image processing, image filtering, triangulation algorithms, geometric relationship processing, registration algorithms, calibration algorithms, and coordinate transformation processing. For example, the POSE of a digitizer probe 338 with an attached probe fiducial marker array 332b may be calibrated such that the probe tip is continuously known as described in U.S. Pat. No. 7,043,961. The POSE of the tool tip or tool axis of the tool 314 may be known with respect to a device fiducial marker array 332a using a calibration method as described in U.S. Prov. Pat. App. 62/128,857. The device fiducial marker 332a is depicted on the manipulator arm 310 but may also be positioned on the base 308 or the tool 314. Registration algorithms may be executed to determine the POSE and coordinate transforms between a bone B, pre-operative bone data, a fiducial marker array 332c and a surgical plan, using the registration methods described in U.S. Pat. Nos. 6,033,415, and 8,287,522.

Upon assembly of the device tracking array 332a to the surgical robot 302 prior to surgery, the POSE's of the coordinate systems, 332a and the end effector tool 314, are fixed relative to each other and stored in memory to accurately track the end effector tool 314 during the surgery (see for example U.S. Patent Publication 20140039517 A1) relative to the target anatomy (e.g., bones B). The POSE data may be used by the computing system 304 during the procedure to update the bone and surgical plan coordinate transforms relative to the tool 314 so the surgical robot 302 can accurately execute the surgical plan in the event any bone motion occurs. It should be appreciated that in certain embodiments, other tracking systems may be incorporated with the surgical system 300 such as an electromagnetic field tracking system or the passive mechanical tracking system 319. An example of a passive mechanical tracking system 319 is described in U.S. Pat. No. 6,322,567. In a particular inventive embodiment, the surgical system 300 does not include a tracking system 306, but instead employs a bone fixation system, a mechanical tracking system 319, and a bone motion monitoring system, where the bone is fixed directly to the surgical robot 302 in the robotic coordinate frame as described in U.S. Pat. No. 5,086,401.

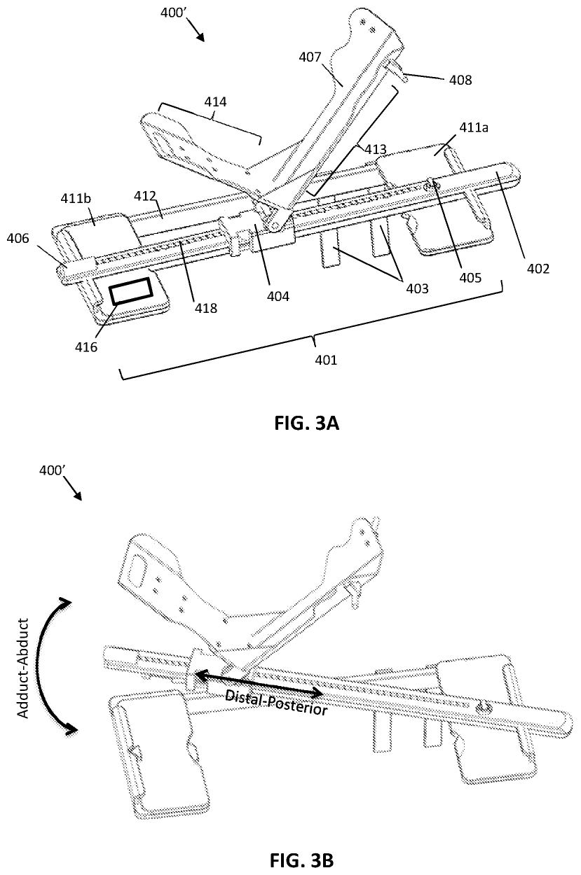

The active anatomical positioning system 400 is configured to automatically position a target anatomy to an optimal position relative to a robot. The anatomical positioning system 400 generally includes an anatomical attachment region and components (e.g., actuators, motors, ball screws, encoders, step motors) in communication with a computer/controller to automatically move the anatomy into an optimal position relative to the surgical robot. Referring to FIG. 3A-3B, a specific embodiment of an anatomical positioning system 400 is an active knee positioning system 400′ for automatically moving the knee or leg bones. The knee positioning system 400′ is configured to interact and communicate with the components of the robotic system 300 to automatically position the knee in an optimal position during a surgical procedure. The knee positioning system 400′ automatically moves the anatomy in at least one degree of freedom to ensure the target anatomy is in the proper position and orientation and according to the optimal position. In more detail, the knee positioning system 400 generally includes a support platform 401, a rail 402, a boot 407, a carriage 404, and one or more actuators (405, 406) in communication with a computer/controller (416, 316, 336).

The support platform 401 is configured to support the boot 407 and assemble the knee positioner system 400′ to a rigid structure such as a hospital bed or surgical robot 302 with one or more attaching elements 403 (e.g., rods, clamps, fastening elements). The support platform 401 may include one or more top frames (411a, 411b), coupled to a side frame 412 to define a rigid structure for the knee positioning system 400′. The support platform 401 is further configured to support and accommodate the rail 402. The rail 402 assembles to the support platform 401 and provides the structure on which the carriage 404 and boot 407 are controllably translated there-along. In one embodiment, the rail 402 is attached or integrated with the one or more top plates (411a, 411b) to remain rigid relative to the support platform 401. In another embodiment, one end of the rail 402 is pivotally coupled to a first top frame 411a, or one end of a single top frame, to allow a second end of the rail 402 to be controllably rotated on a second top frame 411b, or an opposing end of a single top frame.

In general, the boot 407 is configured to secure the distal end of the leg to the knee positioning system 400′. The boot 407 generally includes an upper portion 413 and a heel portion 414, where the foot is rigidly fixed to the heel portion 414 and the leg is fixed to the upper portion 413. A plurality of tapes, straps or any equivalent thereof may be used to wrap around the leg to tightly secure the distal end of the leg to the boot 407. In a particular embodiment, the upper portion 413 further includes one or more fixation elements 408, such as protruding rods, to removably attach and secure the boot 407 to the surgical robot 302 via a series of fixation rods and clamps extending from the robot 302 to connect with the fixation elements 408. The fixation elements 408 may further be attached and secured to a hospital bed via a similar series of fixation rods and clamps. The fixation elements 408 when fixed to the robot or hospital bed provide further stability to the knee during a TKA procedure, essentially reducing motion between the knee and the robotic system during the procedure. However, it is worthy to note, that while the boot 407 is being actuated, the fixation rods are removed from the fixation elements 408 to ensure the boot 407 can move without restriction.

In a specific embodiment, the carriage is configured to assemble the boot 407 to the rail 402 and permit the boot 407 to translate along the rail 402. The carriage 404 is generally a block of metal or other suitable material shaped to conform about the rail 402 and may include a plurality of bearings to allow the movement of the carriage 404 along the rail 402. It should be appreciated that the carriage may be of any shape or material desirable for the purpose of this application.

The knee positioner 400′ further includes one or more actuators (405, 406) and encoders (not shown) to controllably actuate the boot 407. In a particular embodiment, the knee positioner 400′ includes a translational actuator 406 in mechanical communication with the carriage 404 or boot 407 to translate the boot 407 in a distal-posterior direction as shown in FIG. 3B with reference to the distal-posterior motion arrow. The translation actuator 406 may be bi-directional motor or step-motor that rotates a ball screw 418. The carriage 404 may include a ball nut, or have an opening through the carriage in the form of a ball nut. The ball nut is assembled with the ball screw to permit translational motion of the boot 407 upon reversible rotation of the ball screw 418. In a specific inventive embodiment, the rail 402 includes a toothed top surface in mechanical communication with a worm gear associated with the carriage 404. In this embodiment, the translation actuator 406 is housed in the carriage 404 to rotate the worm gear to control the position of the carriage 404 along the length of the rail 402. In another embodiment, the knee positioner 400′ includes a rotational actuator 405 in mechanical communication with the rail 402 to rotate the boot 407 in an adduct-abduct rotational direction as shown in FIG. 3B with reference to the adduct-abduct motion arrow. The rail 402 may be assembled to the actuator 405 at the pivot coupling on the first top plate 411a as described above. The actuators (405, 406) may be bi-directional step motors to incrementally actuate the boot in a step-wise fashion as a safety measure, but it is contemplated that other bi-directional motors or actuators (e.g., pneumatic, electric) may likewise be used. In another embodiment, the plurality of attaching elements 403 may include a translation actuator to permit actuation control in the anterior-posterior direction. Each actuator (405, 406) is assembled and/or in communication with encoders (not shown). The encoders may be relative or absolute and are configured to provide feedback to the computer/controller (416, 316, 306) as to the actuator positions such that the computer/controller (416, 316, 306) can calculate the position of the boot 407 based on the kinematics of the knee positioner 400′. Likewise, the computer/controller (416, 316, 306) may calculate the inverse kinematics to command the actuators to position the boot 407 in a specific position and orientation based on several inputs as further described below.

The knee positioner 400′ may further include features to determine an origin coordinate system of the positioner 400′, where the kinematics of the positioner 400′ are all based from this origin coordinate system. The features may be three divots manufactured in a known position and location on the knee positioner 400′ relative to the actuators (405, 406), encoders, and links of the positioner (e.g., the rail 402). A digitizer may then collect these three points to calculate the origin coordinate system of the positioner 400′ relative to the robot 302 and/or tracking system 306. In another embodiment, the features are a channel and one divot, while in other embodiments the features are a set of characters, lines, and/or fiducial markers manufactured in known position on the positioner 400′ such that an optical tracking system 306 can directly image and calculate the origin coordinate system of the positioner 400′.

It should be appreciated that the knee positioning device 400′ is but one example of an anatomical positioning device 400. Other examples of manual anatomical positioning devices 400 that may be modified with the components as described with the active knee positioner 400′ include the DeMayo knee brace as described in U.S. Pat. No. 7,380,299, the Soloarc knee positioning system (Match Grade Medical LLC, Neenah, Wis.), the TRIMANO® Arm Holder (Arthrex, Inc., Naples, Fla.), the HANA® table (Mizuho OSI, Union City, Calif.), and similar limb positioning devices. Specific inventive embodiments of methods utilizing the robotic system 300 and anatomical positioning device 400 are further described below.

With reference to FIG. 4, an embodiment of an inventive method for automatically positioning a target anatomy (e.g., distal femur and proximal tibia) to an optimal position relative to a robot generally includes the steps of (a) determining a surgical plan, the surgical plan having a task to be executed on a bone (S410); (b) assembling the bone, directly or indirectly, to an anatomical positioning device (S420); (c) evaluating an initial position of the bone relative to the robot (S430); (d) determining an optimal position for the bone based on the initial position and the task (S440); and (e) positioning the bone in the optimal position via automatic movements by the anatomical positioning device (S450). Various embodiments of the method and components are further described in detail below.

The surgical plan may be determined by planning the POSE of the pre-operative bone data relative to the surgical system in a pre-operative planning software program having a graphical user interface (GUI). In a particular embodiment, the pre-operative bone data is a virtual three-dimensional (3-D) bone model generated from an image data set of a subject's anatomy. The image data set may be collected with an imaging modality such as computed tomography (CT), dual-energy x-ray absorptiometry (DEXA), magnetic resonance imaging (MRI), X-ray scans, ultrasound, or a combination thereof. The 3-D bone model(s) are readily generated from the image data set using medical imaging software such as MIMICS® (Materialise, Plymouth, Mich.) or other techniques known in the art such as the one described in U.S. Pat. No. 5,951,475. The user can then save this surgical planning data to an electronic medium that is loaded and read by a robot surgical to assist the surgeon intra-operatively to prepare the bone to receive the physical implant.

The bone may be assembled, directly or indirectly, to the anatomical positioning device 400 by placing a patient's anatomy into the anatomical attachment region (e.g., the boot 407) and strap or otherwise fix the anatomy therein such that there is minimal movement between the bone and any soft tissue. In another embodiment, the anatomical attachment region may be a set of pins that are screwed directly into the bone(s). Once assembled to the positioning device 400, the bone(s) act as additional rigid links in the kinematic chain of the active anatomical positioning device 400. If the target anatomy is a joint, such as a knee joint, the joint may be modeled as a rotational or spherical joint in the kinematic chain. The length and orientation of the ‘bone’ link may be determined by a full or partial registration (described below), or by simply digitizing points on the top of the bone when the bone is secured in the anatomical positioning device 400. Once the length and/or POSE of the ‘bone’ link is known, the kinematic chain of the positioning device 400 is updated in the computer/controller such that the computer/controller can provide commands to the actuators (405, 406) to place the ‘bone’ link and the portion of bone on which the task is to be executed in a specific position, such as an optimal position.

The evaluation of an initial position of the bone relative to the robot may be determined by several methods. In one embodiment, the initial position is determined by fully registering the surgical plan or task to the bone using registration methods known in the art. In another embodiment, a partial registration is performed. The partial registration may include digitizing the very top of the bone where the task is to be performed, such as the most distal surface of the femur between the condyles. Further, the longitudinal axis of the digitizer may be oriented with the long axis of the bone to provide orientation information about the bone. A partial registration may provide enough information to determine an optimal position for the bone and is much faster to execute compared to a full registration. In addition, the evaluation of the initial position of the bone further provides the dimensions of the ‘bone’ link for the anatomical positioning device 400 as described above.

After a partial or full registration of the bone, an optimal position for the bone is determined by one or more computer/controllers (416, 316, 306). The optimal position may be determined based on several algorithms that rely on global and local optimization algorithms. The optimization algorithms may use a kinematic model of the robotic system, and a known POSE of the patient's anatomy (as determined by fully or partially registering the surgical plan to the bone) to determine an optimal position for the bone to achieve the desired reachability within the operative volume such as the points in the cut file, a set of boundaries, or a set of drill holes or planar cuts, defined in the surgical plan. The optimization algorithms may also include additional constraints for determining the optimal position. The constraints may include manipulator requirements such as the avoidance of a singularity, a joint limit, or a collision of the manipulator arm while executing the surgical plan. The constraints may include line-of-sight considerations where the location of a fiducial marker array relative to the tracking system may be optimized for a particular bone and base position or manipulator arm configuration. The constraints may further include user's preferences for the position of the bone, to provide the user with particular access points or corridors to the operational site, where the robot is still capable of executing the surgical plan. The preferences may also include how the bone or base should be oriented to easily grasp and wield the manipulator arm or end-effector tool if a passive or haptic surgical robot is used. The algorithm constraints may also include patient factors such as the patient's body mass index (BMI), the operating side (e.g., left or right femur), or amount of exposure of the targeted anatomy. In a particular embodiment, a user may simply choose the optimal position for the bone relative to the robot in the pre-operative planning software.

Finally, once the optimal position is determined, the bone is positioned in the optimal position via automatic movements by the anatomical positioner 400. The kinematics of the anatomical positioner 400 are updated with the new ‘bone’ link parameters, the origin coordinate system is determined relative to the robot 302 and/or tracking system 306, and the actuator positions are read via the encoders to fully define the forward kinematics of the positioning device 400 and the initial position of the target bone. Subsequently, the computer/controller (416, 316, 306) may then calculate the inverse kinematics to command the actuators (405, 406) to position the bone in the determined optimal position. It should be appreciated, that determining the origin coordinate system and therefore the POSE of the knee positioning system 400′ relative to the robot 302 or tracking system 306 may be performed at any point prior to the automatic movements to position the knee in the optimal position.

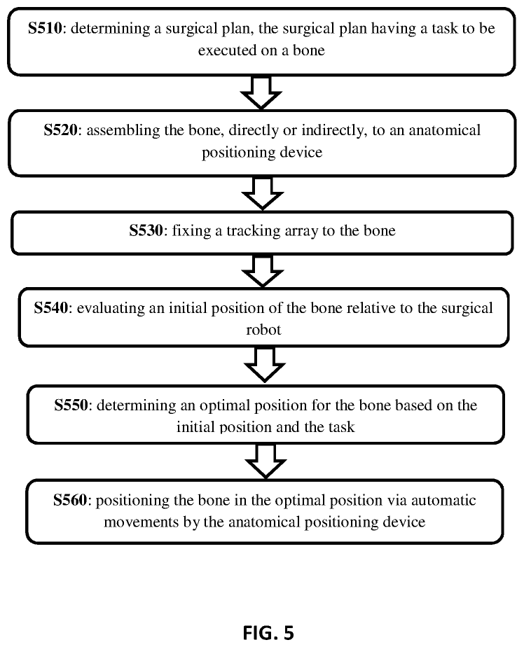

With reference to FIG. 5, a specific inventive embodiment of a method using the knee positioning system 400′ is shown. The method is performed with a robotic system including a surgical robot 302, an optical tracking system 306, and the knee positioning device 400′ having a tracking array 332c. The method in more detail is as follows: (a) determining a surgical plan, the surgical plan having a task to be executed on a bone (S510); (b) assembling the bone, directly or indirectly, to an anatomical positioning device (S520); (c) fixing a tracking array to the bone (S530); (d) evaluating an initial position of the bone relative to a surgical robot (S540); (e) determining an optimal position for the bone based on the initial position and the task (S550); and (f) positioning the bone in the optimal position via automatic movements by the anatomical positioning device (S560).

The method of FIG. 5 may further include monitoring the position of the knee, in real-time, relative to the optimal position. If the position of the knee is at or beyond the optimal position, feedback is transferred to the positioning computer/controller 416, device computer 316 and/or tracking computer 306 through a wired or wireless connection. The computer/controller (416, 316, 306) may then command the actuators (405, 406) to automatically move the knee, continuously or intermittently to the optimal position based on the feedback.

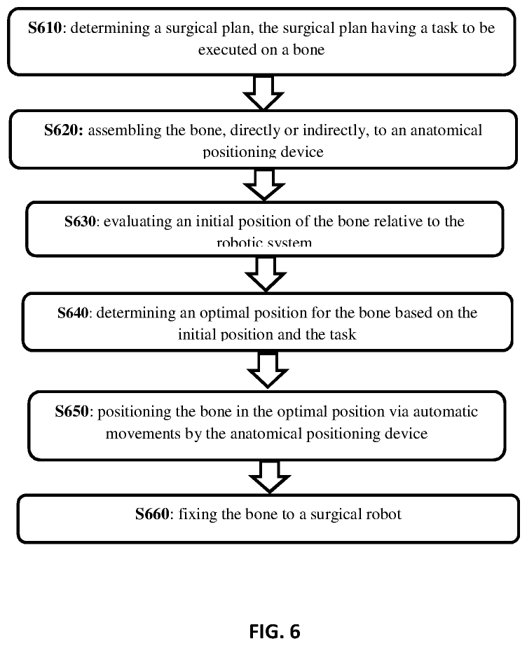

Referring to FIG. 6, a specific inventive embodiment of a method using the knee positioning system 400′ is shown. The method is performed with a surgical system including a surgical robot 302 equipped with a bone fixation and monitoring system and the knee positioning device 400′. The method in more detail is as follows: (a) determining a surgical plan, the surgical plan having a task to be executed on a bone (S610); (b) assembling the bone, directly or indirectly, to an anatomical positioning device (S620); (c) evaluating an initial position of the bone relative to the surgical robot (S630); (d) determining an optimal position for the bone based on the initial position and the task (S640); (e) adjusting the leg fixture to position the bone in the optimal position (S650); and (f) fixing the bone to a surgical robot via a series of fixation rods and clamps such that the bone remains in the optimal position throughout the procedure (S660).

In a particular embodiment, the robotic system 302 includes an upper leg anatomical positioning device having an upper leg attachment region for attachment to a patient's upper leg, and similar components and actuating elements as the knee positioner 400′. A particular embodiment of a method performed with a robotic system including a surgical robot 302, an optical tracking system 306, and the knee positioning device 400′ having a tracking array 332c, and upper leg anatomical positioning device may include: (a) determining a surgical plan, the surgical plan having a task to be executed on a bone; (b) assembling a first bone, directly or indirectly, to an anatomical positioning device; (c) assembling a second bone, directly or indirectly, to an anatomical positioning device; (d) fixing a tracking array to the first bone and second bone; (e) evaluating an initial position of the first bone and second bone relative to a surgical robot; (f) determining an optimal position for the first bone and second bone based on the initial positions and the task; (g) positioning the first bone and second in the optimal position via automatic movements by the knee positioner and the upper leg positioner, respectively; and (f) rejoining or articulating the first bone and the second bone, via automatic movements by the knee positioner and upper leg positioner, to a planned position after modifications have been made to either the first bone or second bone to at least: 1. assess the quality of the procedural modification; 2. aid in assembling implant components to first bone and/or second bone; and 3. assess the balance of one more ligaments connected between the first bone and second bone.

Coordinate Transformations for Anatomical Positioning System with a Surgical Robot

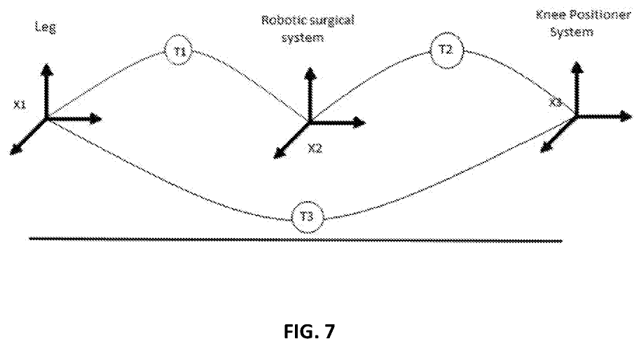

With reference to FIG. 7, to generally perform the methods described herein, several coordinate transformations need to be determined and/or calculated. FIG. 7 depicts several different coordinate systems associated with the system and methods described herein including a first coordinate system X1 associated with the leg (L), a second coordinate system X2 associated with the knee positioner system 400′, and a third coordinate system X3 associated with the surgical robot/tracking system.

A first coordinate transformation T1 is a transformation from the leg coordinate X1 to the surgical robot coordinate system X2. T1 is calculated and fixed after the user fully or partially registers the bone when the bone is attached to the knee positioner system 400′. A second coordinate transformation T2 is a transformation from the knee positioner coordinate system X2 to the surgical robot coordinate system X3. The second coordinate transformation T2 may be determined by digitizing three or more points manufactured on the knee positioner system as described above. The third coordinate transformation T3 is a transformation from the knee positioner coordinate system X2 to the leg coordinate system X1, where T3 is calculated as follows:

_{X1}T1^{X2}·_{X2}T2^{X3}=_{X3}T^{X1} a.

The transformation T³ may be used to define the ‘bone’ link in the kinematic chain of the positioning device 400′.

### Other Embodiments

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient roadmap for implementing the exemplary embodiment or exemplary embodiments. It should be understood that various changes can be made in the function and arrangements of elements without departing from the scope as set forth in the appended claims and the legal equivalents thereof.

The foregoing description is illustrative of particular embodiments of the invention, but is not meant to be a limitation upon the practice thereof. The following claims, including all equivalents thereof, are intended to define the scope of the invention.

1. A robotic surgical system for performing a surgical procedure on a target anatomy of a subject comprising:
a computing system configured to generate surgical plan data;
a surgical robot having a movable base connected to a manipulator arm and adapted to hold an operating tool, said surgical robot receiving positional information associated with the surgical plan data from said computing system; and
an active anatomical positioning device having an anatomical attachment region adapted to couple to the subject and in communication with a controller, said anatomical positioning device adapted to automatically position the target anatomy relative to the surgical robot in response to instructions from said computing system.

2. The surgical robot system of claim 1 further comprising a tracking system, said tracking system further comprising at least two optical receivers configured to detect a position of a plurality of fiducial markers.

3. The surgical robot system of claim 1 further comprising a mechanical digitizer arm.

4. The surgical robot system of claim 1 wherein the computer system comprises a planning computer configured to generate the surgical plan data, a device computer configured to control said surgical robot, a positioning device computer configured to control said anatomical positioning device, and at least one peripheral device.

5. The surgical robot system of claim 4 wherein the surgical plan data includes operational task data for modifying tissue and bone relative to the target anatomy.

6. The robotic surgical system of claim 1 where the active anatomical positioning device comprises:
a support platform having a top frame coupled to a side frame;
a rail supported by the support platform;
a carriage slidably mounted to the rail;
a boot having a upper portion and a lower portion configured to receive and retain a distal end of an anatomical extremity, wherein the boot is attached to the carriage; and
a first actuator in communication with the controller to controllably actuate the boot along the rail via the carriage.

7. The robotic surgical system of claim 6 wherein the support platform comprises fastening elements configured to attach the active anatomical positioning device to one of a bed or the surgical robot.

8. The robotic surgical system of claim 6 wherein the rail is pivotally coupled to the top frame of the support platform and the active anatomical positioning device further includes a second actuator in communication with the controller to pivot the rail in an adduct-abduct rotational direction relative to the top frame.

9. The robotic surgical system of claim 6 wherein the active anatomical positioning device further comprises an encoder configured to provide positional feedback to the computing system regarding a position of the boot.

10. The robotic surgical system of claim 6 wherein the active anatomical positioning device further comprises at least one origin feature in a known position relative to the rail.

11. The robotic surgical system of claim 10 further comprising a digitizer configured to digitize the origin feature to permit the computing system to determine an origin coordinate system of the active anatomical positioning device relative to a surgical robot or tracking system.

12. The robotic surgical system of claim 10 wherein the origin feature is a divot, a channel, a fiducial marker, or a combination thereof.

13. A method for positioning a target anatomy of a subject in an optimal position relative to a robotic surgical device of claim 1, the method comprising:
determining a surgical plan based on surgical plan data, the surgical plan having a task to be executed on the target anatomy;
coupling the subject proximal to the target anatomy to said anatomical attachment region;
evaluating an initial position of the target anatomy relative to said robotic surgical system;
determining the optimal position for the target anatomy based on the initial position and the task; and
positioning the target anatomy in the optimal position by adjusting a position of the boot with the active anatomical positioning device relative to the initial position to the optimal position prior to executing the task.

14. The method of claim 13 wherein the surgical plan is determined by a planning software on said computing system.

15. The method of claim 13 wherein the surgical plan is determined by evaluating bone data of the target anatomy.

16. The method of claim 13 wherein the optimal position is determined by the computing system.

17. The method of claim 13 wherein determining the optimal position comprises assessing a reachable extent of the surgical robot based on the surgical plan and the determined initial position of the bone.

18. The method of claim 13 wherein the position of the positioning device is adjusted by communication between said computing system and said controller.

19. The method of claim 13 wherein the target anatomy is a knee and said positioning is flexure of said knee.

20. The method of claim 13 further comprising monitoring a position of the target anatomy on a time scale of at least once every second.
